(19) United States
(12) Patent Application Publication
(10) Pub. No.: US 2012/0142052 A1
(43) Pub. Date: Jun. 7, 2012

(54) METHOD AND DEVICE FOR MULTIPLE FEED CULTIVATION

(76) Inventors: Christian Kohlschovsky, Paehl (DE); Rolf Rotenburg, Grosshesselohe (DE)

(21) Appl. No.: 13/389,659

(22) PCT Filed: Aug. 11, 2010
(86) PCT No.: PCT/EP10/04917

§ 371 Date: Feb. 9, 2012

(30) Foreign Application Priority Data
Aug. 13, 2009 (EP) 09010426.6

(51) Int. Cl. C12P 21/00; C12N 5/00; C12N 1/00; B01L 9/00; C12M 1/04

(52) U.S. Cl. 435/69.1; 422/566; 435/289.1; 435/243; 435/325

(57) ABSTRACT

The current invention is directed to support units for mounting a cell cultivation liquid storage unit which can be connected to a balance, a cell cultivation apparatus using such a support unit, and the use of such a support unit in a method for the cultivation of cells.

The current invention is directed to a method and a device useful in this method for the cultivation of prokaryotic and eukaryotic cells allowing for a high degree of flexibility in arranging and performing the cultivation.

### BACKGROUND OF THE INVENTION

Therapeutic proteins account for a large part of today's medical portfolio. The need of these therapeutic proteins cannot be fulfilled with conventional production methods. In order to provide the required large amount of the therapeutic proteins highly elaborated recombinant production methods are employed.

Recombinant production of therapeutic proteins is generally carried out in cultivation vessels. When looked upon, such a cultivation vessel is a complex entity, which has e.g. to be sterilized, tempered, stirred and aerated. A multitude of different pipes, heat exchange devices, peristaltic pumps, valves and the like are required for a proper performance of the vessel during the cultivation process.

The entire cultivation process, including the past cultivation cleaning processes is performed according to guidelines permitting only a limited deviation from pre-specified conditions. To ensure the conformity of the processes a multitude of devices in the production process have to be controlled and a multitude of parameters have to be determined and recorded.

For the cultivation of prokaryotic and eukaryotic cells different cultivation methods can be used, such as cultivation without feeding, fed-batch cultivations or continuous cultivations. In fed-batch cultivation e.g. a change in the pH value triggers the addition of an acidic or alkaline solution in order to adjust the pH value. In general a fed-batch cultivation method requires a defined feedback control in order to control the supply of required components. This is achieved by the use of a feed of solutions in order to maintain process parameters at predetermined set points or in predetermined ranges.

The fed-batch cultivation method is at the moment preferred for large scale cultivations. Beside the addition of essential, i.e. consumed by the cultivated cells, compounds to the cultivation medium other process parameters, such as the pH value, are kept within a predetermined range. The compliance of certain process parameters with predetermined ranges is of importance. Non-optimal process parameters do not only interfere with the productivity of the cultivated cell but also with the quality and homogeneity of the produced therapeutic protein. Thus, the control of the relevant process parameters as well as a readjustment of the parameters has to be performed.

The thereby required technical complexity has reached a barley acceptable level for basic maintenance and repair work required during the cultivation processes.

If the cultivation requires a specific feeding strategy with multiple solutions, e.g. different nutrient feed solutions, a base and an acid for pH control, an anti-foam agent, and the like, the spatial arrangement of the required storage units for these solutions cannot be achieved without interfering with maintenance work during the cultivation process.

In EP 1 266 960 a method an apparatus for culturing cell or tissue is reported. A method for metering nutrient media to cell culture containers is reported in U.S. Pat. No. 4,937,194. In EP 1 092 764 continuous fermentation is reported. A weighting apparatus is reported in GB 2 229 823. In Japanese patent application publication JP 2000-262372 an implement for attaching a frame is reported. An apparatus for comparing the leverage-effects upon the hands, of sporting and athletic implements is reported in GB 252,658. In Japanese patent application publication JP 2005-287425 a culture bag with culture medium bag is reported.

### SUMMARY OF THE INVENTION

An aspect as reported herein is a support unit, e.g. for mounting a cell cultivation liquid storage unit below a carrier unit, characterized in that, the support unit comprises at one end an eye (1) for mounting the support unit to the carrier unit and at the other end a hook (5) for taking up the cell cultivation liquid storage unit, which are connected by a rod having no offset in the section (2) connected with the eye and having an offset (4) in the section (3) connected with the hook, whereby the offset in the section (3) connected with the hook is of from 5 A.U. to 50 A.U. with respect to the rod in the section (2) connected with the eye.

It has been found that the support units as reported herein reduce the spatial requirements of storage devices used in cultivation processes having the requirement of the provision of different feed solutions. With the devices as reported herein the liquid storage units can be mounted above the cultivation vessel and therewith the direct access to the cultivation vessel is eased. At the same time the support unit as reported herein allows for the mounting of up to five different liquid storage units on a single support unit. Also at the same time it is possible to mount bag-type and bottle-type cell cultivation liquid storage units to the same support unit. It has also been found that by providing an additional means (9) the use of the storage unit can be made even more flexible. By upgrading the support unit with the further means (9) it is on the one hand possible to support large bag-type liquid storage units that have been mounted to means (701) or if a small bag-type storage unit is mounted to means (701) to mount two further bag-type liquid storage units. The support of a large bag-type liquid storage unit by means (9) prevents the handle with which the bag-type liquid storage unit is mounted to the support unit from disrupting.

To be more precise, herein is reported a support unit for placing a cell cultivation liquid storage unit below a carrier unit, characterized in that, the support unit comprises
a first means (1) at the upper end (100) of the support unit for mounting the support unit to the carrier unit,
a second means (5) at the lower end (200) of the support unit for mounting the cell cultivation liquid storage unit to the support unit, and
a solid connection (103) for connecting the first means (1) to the second means (5) comprising a first straight section (2),
whereby the first means (1), the first straight section (2) and the balance point of the support unit once the cell cultivation liquid storage unit has been mounted are in line.

In one embodiment the unit further comprises a first straight section (2), a second straight section (3) and an interjacent offset section (4),
whereby
the axis (101) of the first straight section (2) is parallel to the axis (201) of the second straight section,
the offset in the offset section (4) between the axis (101) of the first straight section (2) and the axis (201) of the second straight section (3) is of from 5 to 50 A.U.,
the offset section (4) has the same angle (102) with respect to the axis of the first straight section (101) and the axis of the second straight section (201).

In another embodiment the first means is selected from an eye and a clamp. In also an embodiment the second means is selected from a hook, a plate and a flange. In a further embodiment the second means has a shape selected from circle, square, rectangle, ellipse, trapezium, parallelogram, and rhombus.

In one embodiment the unit is further characterized in that
the offset between the axis (101) of the first straight section (2) and the axis (201) of the second straight section (3) is 10 A.U.,
the first means is an oval eye for taking up a hook of the carrier unit and has a length to diameter ratio of about 2:1,
the straight sections (2) and (3) have either the same or a different length,
the angle (102) of the offset section (4) is of from 90 degrees up to 160 degrees,
the second means (5) has an angle (202) of 90 degrees with respect to the axis (201) of the second straight section (3).

In one embodiment the support unit is further characterized in that
the first means is an oval eye for taking up a hook of the carrier unit and has a length to diameter ratio of about 2:1 wherein the length is of from 10 to 16 A.U. and a diameter of from 5 to 8 A.U., and/or
the straight sections (2) and (3) have both the same length of from 72 A.U. to 138 A.U., and/or
the offset section (4) has a length of from 8 A.U. to 12 A.U., and/or
the second means (5) has a distance of from 50 to 60 A.U. to the second bend of the offset (4).

Also an aspect is a support unit for mounting one to three cell cultivation liquid storage units below a carrier unit, characterized in that the support unit comprises
a central component (6) with an upper flange (14), a lower flange (13) and a height (302),
a first means (7) and a second means (701) each for mounting a bag-type cell cultivation liquid storage unit,
a third means (8) for mounting a bottle-type cell cultivation liquid storage unit,
a block-out (301) close to the upper flange (14) of the central component (6) for mounting the support unit to the carrier unit,
whereby
the central component (6) has the outer shape of an isosceles trapezoid with a width at the upper flange (14) that is less than half the width at the lower flange (13),
the first means (7) and the third means (8) are attached on the same side of the central component (6), and the first means (7) and the second means (701) are attached on opposite sides of the central component (6),
the first means (7) and the second means (701) are attached each within a distance of at most one third of the height (302) of the central component (6) from the upper flange (14) and the third means (8) is attached within a distance of at most one third of the height (302) of the central component (6) from the lower flange (13).

In one embodiment the unit is further characterized in that
the central component (6) comprises an isosceles trapezoid-like block-out (303), and/or
the first means (7) and the second means (701) have the same shape and size and are attached opposite of each other at each side of the central component (6), and/or
the first means (7) and the second means (701) both have an L-like shape whereby the face (307) which is attached to the central component (6) is perpendicular to the face of the central component (6) and the other face (306) is parallel to the face of the central component (6), and/or
the third means (8) comprises
   i) a basis plate (10) with the shape of a segment of a circle with a width (501) of more than the radius of the corresponding circle (502),
   ii) two upward teeth (11) extending to the upper flange (14) of the central component (6) with an angle (305) of 90 degree with respect to the basis plate (10),
   iii) a support plate (12) attached i) to the bottom of basis plate (10) and ii) to the lower flange (13) of the central component (6),
   whereby the basis plate (10) is attached to the central component (6) with an angle (304) of 75 to 85 degrees, and/or
the central component (6) further comprises a fourth means (9) that
   i) is attached to the lower flange (13) of the central component (6),
   ii) is directed to the opposite side of the first means (7) as well as the third means (8),
   iii) is attached to the lower flange (13) with an angle (601) of 130 to 150 degrees, and is bend three times upward in direction to the upper flange (14) with angles (602), (603) and (604) of 150 to 165 degrees, 120 to 140 degrees, and 120 to 140 degrees, respectively.

In another embodiment the unit is further characterized in that
the central component (6) has
   a lower flange (13) length of from 290 to 320 A.U.,
   an upper flange (14) length of from 90 to 110 A.U.,
   an outside flange (15) length of from 530 to 570 A.U., and/or
the first means (7) and the second means (701) are attached at a distance of 150 to 170 A.U. from the upper flange (14) of the central component (6) and has a basis plate length (307) of 25 to 35 A.U. and an upward height (306) of 10 to 14 A.U., and/or
the third means (8) which is attached in the lower third of the central component (6) has a basis plate (10) height of 120 to 140 A.U., a radius of 90 to 100 A.U., a tooth (11) height of 50 to 60 A.U., a tooth width of 40 to 50 A.U., and the support plate (12) has a length of 70 to 100 A.U. and an angle to the basis plate (10) of 140 to 160 degrees, and/or
the fourth means (9) has a first distance (16) of 180 to 200 A.U. from the lower flange (13) to the first upward bend (20), a second distance (17) of 130 to 150 A.U. from the first upward bend (20) to the second upward bend (21), a third distance (18) of 60 to 75 A.U. from the second upward bend (21) to the third upward bend (22), and a fourth distance (19) of 110 to 130 A.U. from the third upward bend (22) to the end of the fourth means (23).

Another aspect as reported herein is a device comprising a support unit for mounting one to three cell cultivation liquid storage units below a carrier unit comprising a fourth means (9) and a large bag-type cell cultivation liquid storage unit.

In one embodiment the carrier unit is selected from an electronic balance with a hook at its bottom, a mechanical balance, a moveable scaffold with fixed height of more than the overall length of the support unit, or a hook.

In one embodiment the fourth means (9) has a U-like shape. In another embodiment the central component is planar and has a plate-like shape.

Also an aspect as reported herein is a device comprising a support unit as reported herein and a carrier unit selected from an electronic balance with a hook at its bottom, a mechanical balance, a moveable scaffold with fixed height of more than the overall length of the support unit, or a hook.

Also reported herein is the use of the support unit as reported herein for taking up a cell cultivation liquid storage unit and mounting the support unit to a carrier unit whereby the support unit is freely suspended below the carrier unit.

In one embodiment 1 A.U. is 1 mm or 1 cm or 1 in.

Another aspect as reported herein is a cell cultivation apparatus comprising
a) a cell culture vessel,
b) a gas supply,
c) at least cell cultivation liquid storage units for an acidic solution, a basic solution, an anti-foam solution, and a nutrient solution,
d) at least sensors for determining the concentration of oxygen, the concentration of carbon dioxide, the pH value, and the temperature in the cell culture vessel,
e) a control unit for recording the sensor data and controlling the addition of the solutions of c) to the cell culture vessel,
f) at least one pump for the addition of the solutions of c) to the cell culture vessel under control of the control unit e),
   whereby
   up to five storage units are connected to one balance,
   at least one of the storage units connected to the balance is placed below the balance in a support unit as reported herein, and
   the end of the addition of each of the solutions to the cell cultivation vessel is triggered by the weight loss of the respective liquid storage unit from the beginning of the addition.

In one embodiment the balance is placed at 1.5 meter to 4.0 meter above the floor of the room in which the cell cultivation apparatus is operated. In also an embodiment the liquid storage units are connected via flexible, plastic pipes with the pump and the pump is connected with the cell culture chamber via a flexible, plastic pipe.

Also an aspect as reported herein is a method for the cultivation of cells using a cell cultivation apparatus as reported herein.

Further reported herein is a method for producing a polypeptide comprising the following steps:
a) providing a cell containing a nucleic acid encoding the polypeptide,
b) cultivating the cell in a cell cultivation apparatus as reported herein,
c) recovering the polypeptide from the cultivation medium or the cells,
d) optionally purifying the polypeptide,
   and thereby producing a polypeptide.

### DETAILED DESCRIPTION OF THE INVENTION

The current invention is directed to support units for mounting cell cultivation liquid storage units e.g. to a balance, a cell cultivation apparatus using such support units, and the use of such support units in a method for the cultivation of cells.

In fed-batch cultivation methods generally a specific feeding strategy with multiple solutions, e.g. different nutrient feed solutions, a base and an acid for pH control, an anti-foam agent, and the like, is employed. The spatial arrangement of the required liquid storage units cannot be achieved without interfering with the accessibility of the cultivation equipment, such as the cultivation vessel, during the cultivation.

It has been found that the support units as reported herein reduce the spatial requirements of storage devices in cultivation processes requiring the provision of different feed solutions. With the support units as reported herein different liquid storage units can be combined and mounted above the cultivation vessel e.g. to the ceiling of the room, and therewith direct access to the cultivation vessel is eased.

At the same time a support unit as reported herein allows for the mounting of up to five different liquid storage units to a single support unit. At the same time it is also possible to mount bag-type and bottle-type cell cultivation liquid storage units to the same support unit.

The term “bag-type cell cultivation liquid storage unit” denotes a single use flexible container for sterile fluid containment. The storage unit comprises a handle for mounting it to the support unit or transportation. The bag-type storage unit is in general made of a multi-layer polymeric film. The outermost structural or abuse layer can be polyethylene, (oriented) polyester, or (oriented) polypropylene, but can also include (oriented) nylon. This layer preferably is (reverse) printable and advantageously unaffected by the sealing temperatures used to make the bag and compartments, since the bag is sealed through the entire thickness of the multilayer structure. The thickness of this layer can be selected to control the stiffness of the bag, and may range from about 10 to about 60 μm, preferably about 50 μm. The inner layer can include one or more barrier layers, depending on which atmospheric conditions (oxygen, humidity, light, and the like) that potentially can affect the product inside the bag. Barrier layers can be metalized (oriented) polypropylene (PP), aluminum foil, (oriented) polyethylene terephthalate (PET), ethylene vinyl alcohol (EVOH), nylon or (biaxial oriented) nylon, blends or composites of the same as well as related copolymers thereof. Barrier layer thickness will depend on the sensitivity of the product and the desired shelf life. The innermost layer of the package is the sealant. The sealant is selected to have minimum effect on the contents, to be unaffected by the product, and to withstand sealing conditions (such as liquid droplets, grease, dust, or the like). Sealants used in bags or sachets can include ethylene copolymers, such as low density polyethylene (LDPE), linear low density polyethylene (LLDPE), metallocene polyethylene, or copolymers of ethylene with vinyl acetate or methyl acrylate or copolymers of ethylene and acrylic acid (EAA) or methacrylic acid (EMAA), optionally ionomerized (i.e., partially neutralized with metal ions such as Na, Zn, Mg, or Li). Sealants can also include polypropylene copolymers. Sealant layers can be 25 to 100 μm thick. The term “large” denotes a volume of more than 5 liter, in one embodiment more than 10 liter, in also an embodiment more than 20 liter, and in a further embodiment more than 10 liter and less than 100 liter.

The term “bottle-type cell cultivation liquid storage unit” denotes vessel made of glass, or plastic, or metal, used for storing solutions used during the cultivating of prokaryotic and eukaryotic cells. A bottle-type storage unit typically is cylindrical without handles and with a neck that has a smaller diameter than the body of the bottle.

It has also been found that by providing an additional means (9) the use of the storage unit can be made even more flexible and further the reliability of the support unit can be increased, especially when large bag-type storage units are mounted.

By adding to the support unit comprising a central component (6) a further means (9) it is on the one hand possible to support large bag-type liquid storage units that have been mounted to means (701) or on the other hand if a small bag-type storage unit is mounted to means (701) it is possible to mount two further bag-type liquid storage units. The support of a large bag-type liquid storage unit by means (9) e.g. prevents the handle with which the bag-type liquid storage unit is mounted to the support unit from disrupting.

The addition of solutions by weight to a cultivation medium allows for a more efficient utilization of the limited space available if the support units as reported herein are employed. With a weight controlled addition it is possible to control and monitor the addition of up to five different solutions by employing a single balance and using a discontinuous dosing method.

It is advantageous to attach the balance(s) with the mounted support units containing the liquid storage units to the ceiling of the room in which the cell cultivation is performed. With an attachment to the ceiling the access to the cell cultivation device and the related technical equipment is easily possible as the operator can walk below the balance and access the cultivation apparatus for maintenance, repairing, checking, and handling. Furthermore the balance and the liquid storage units are shielded from the vibrations of the agitator of the cultivation vessel. This can further be improved by the use of flexible plastic pipes and connectors. Furthermore, by using flexible bags as liquid storage units and the support units as reported herein the complete solution can be added to the cultivation.

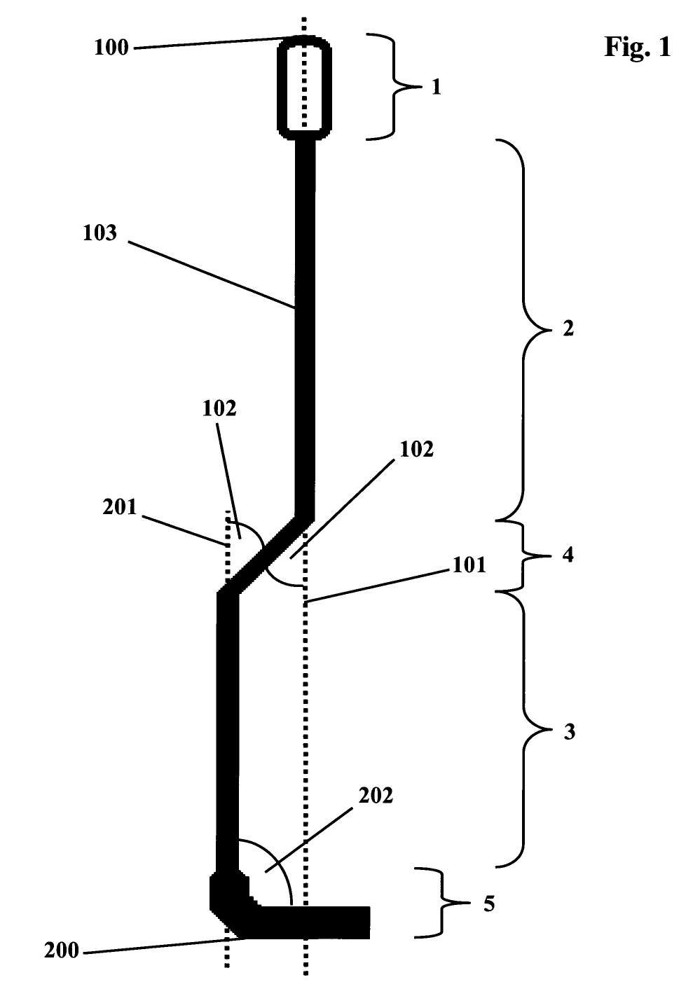

In FIG. 1 a schematic drawing of a support unit as reported herein is shown. The support unit comprises a first means in form of an eye (1), a first straight section (2) connected to the eye, a second straight section (3) connected to a second means in the form of a hook, an offset section (4) and a hook (5).

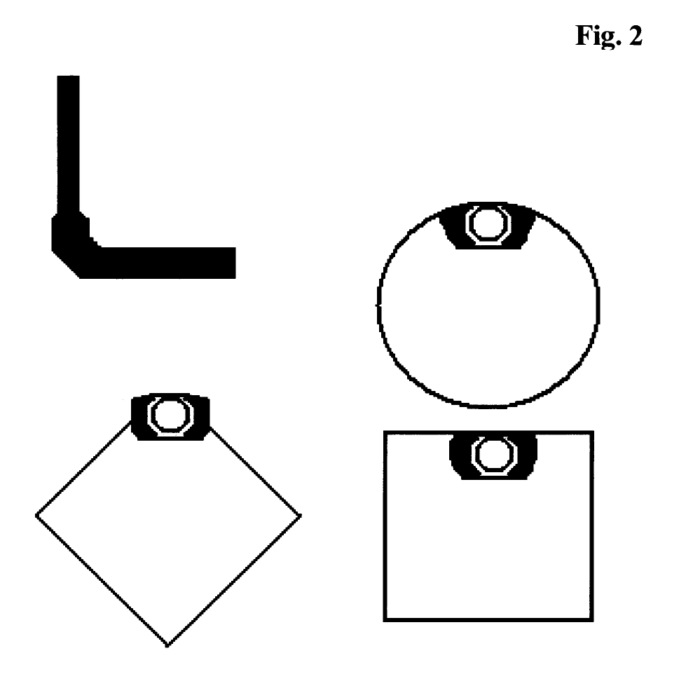

FIG. 2 shows different embodiments of the second means viewed along the axis (101) except for the upper left corner in which the view is perpendicular to the axis (101) and (201).

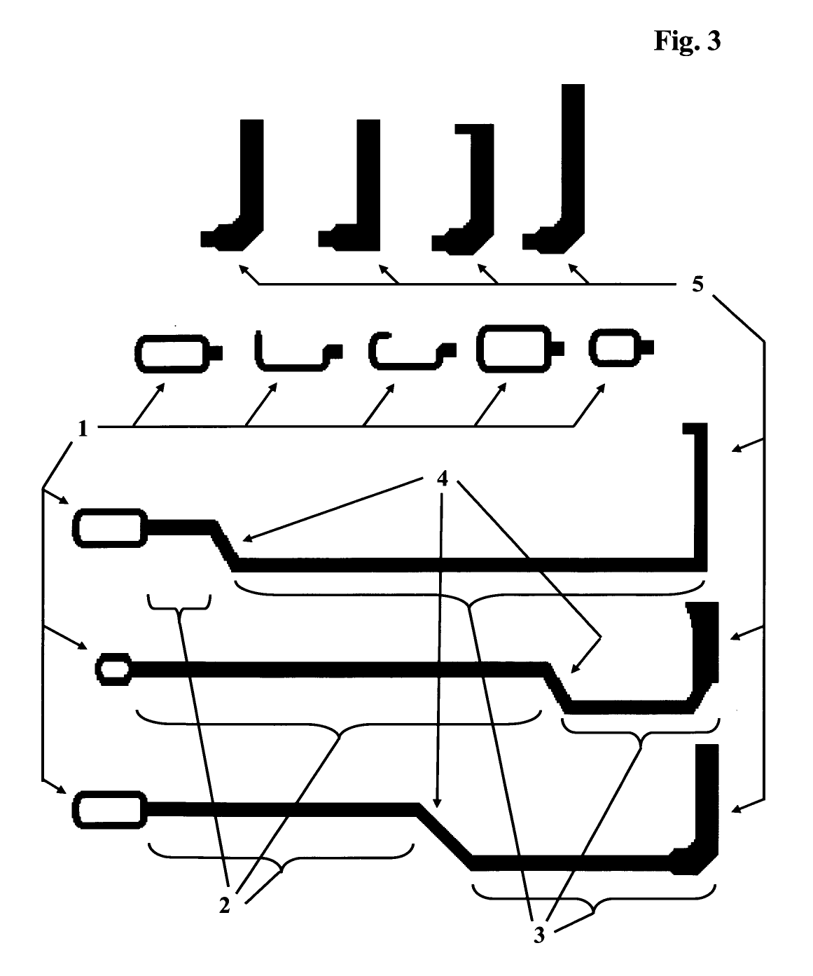

In FIG. 3 different embodiments of the first means (1), the first straight section (2), the second straight section (3), the offset section (4), and the second means (5) are shown.

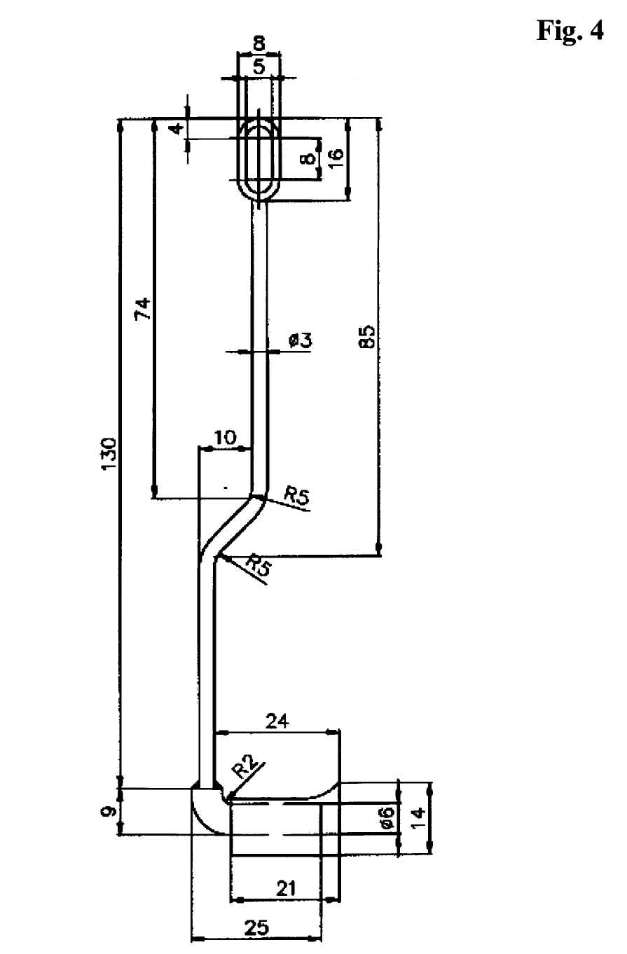

In FIG. 4 the scheme of a specific embodiment of a support unit as reported herein is shown, wherein the numbers given denote diameters and lengths in A.U.

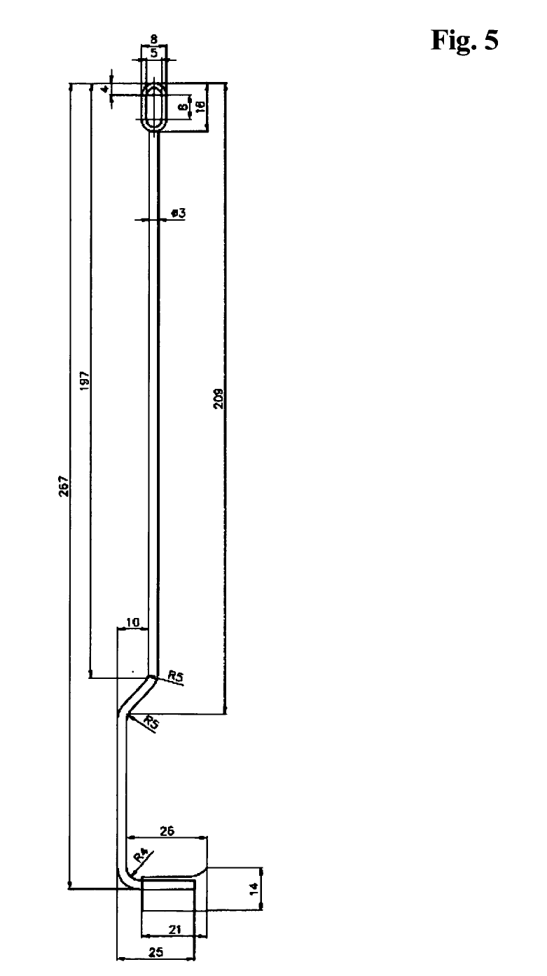

FIG. 5 shows a further specific embodiment of a support unit as reported herein. The numbers given denote diameters and lengths in A.U and R5 denotes 135 degrees.

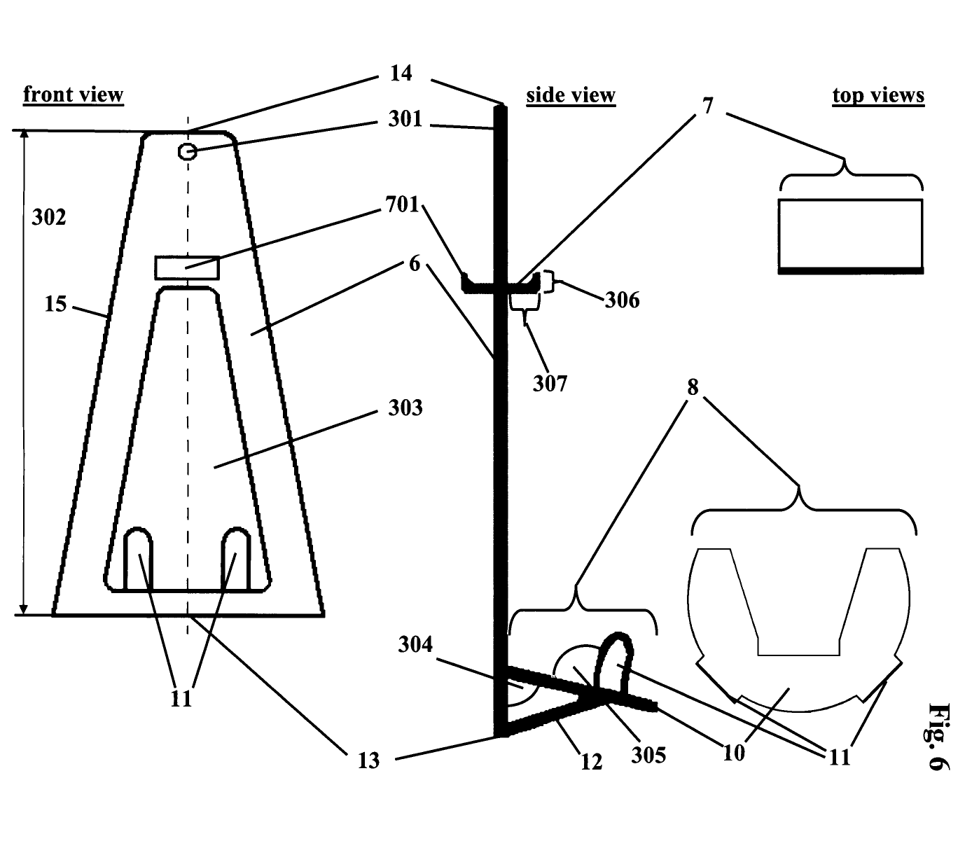

FIG. 6 shows the scheme of a further support unit as reported herein comprising a central component (6), a first means (7) and a second means (701), a third means (8), a support plate 12, a lower flange (13), an upper flange (14) and an outside flange (15).

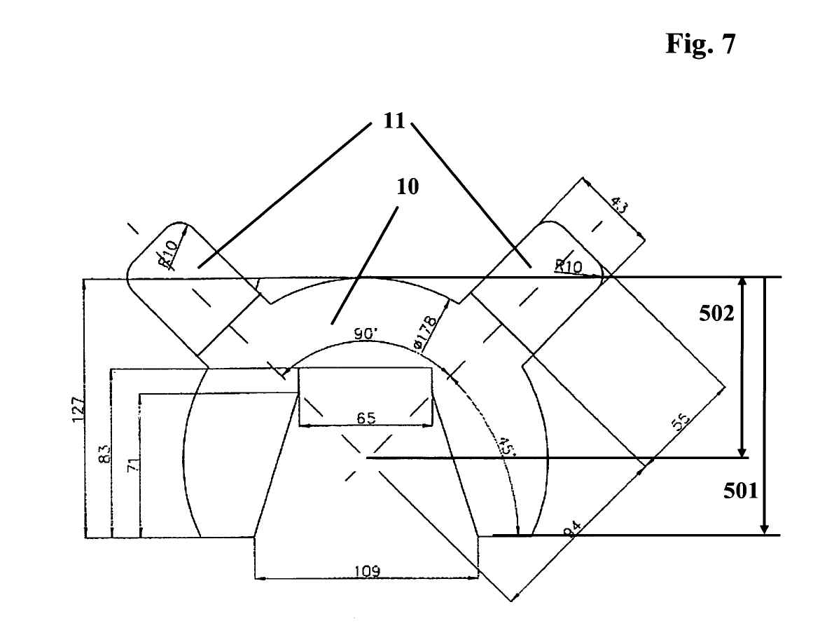

FIG. 7 shows a top down scheme of the third means (8) with the basis plate (10) and the teeth (11).

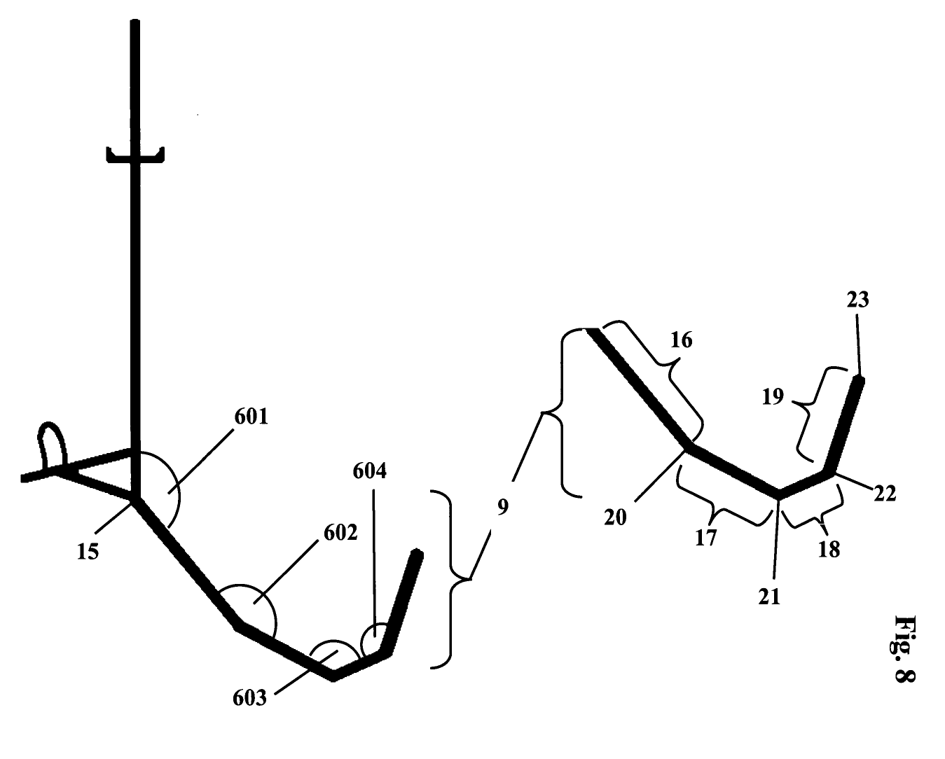

In FIG. 8 the support unit comprising the fourth means (9) as well as the single fourth means (9) is shown. The fourth means comprises the distance (16) from the lower flange (13) to the first upward bend (20), the distance (17) from the first upward bend (20) to the second upward bend (21), the distance (18) from the second upward bend (21) to the third upward bend (22), and the distance (19) from the third upward (22) bend to the end of the fourth means (23).

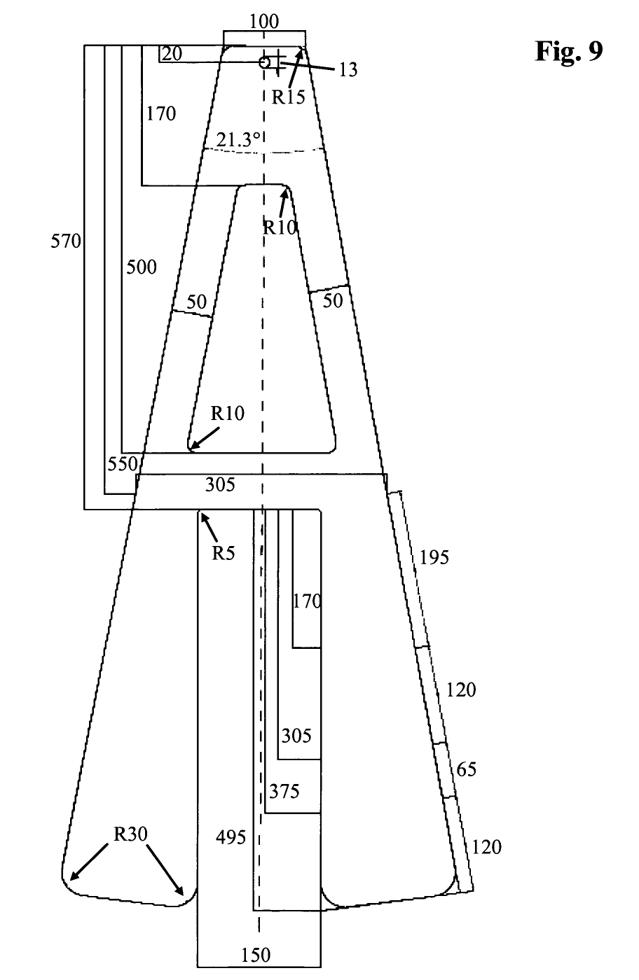

FIG. 9 shows a front view of an embodiment of the support unit as reported herein seen from the side of the second means (701) and the fourth means (9). The numbers given denote diameters and lengths in A.U.

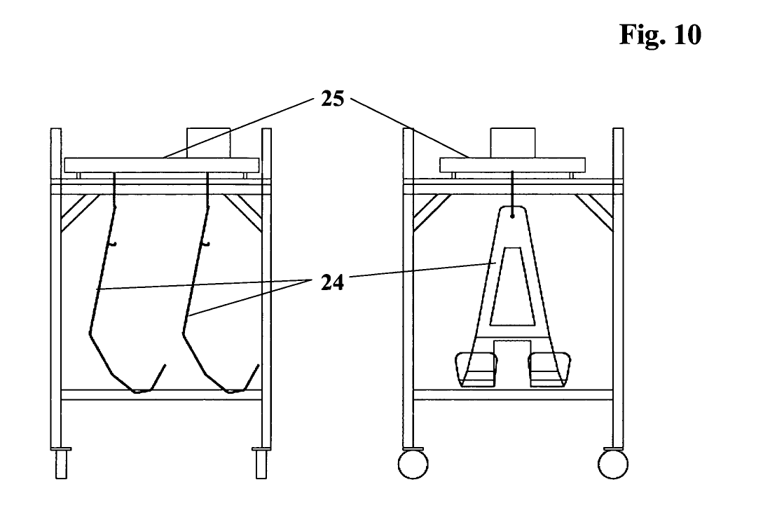

FIG. 10 shows an exemplary application of a support unit (24) as reported herein mounted below a balance (25) on a mobile laboratory as side view (left) and as front view (right).

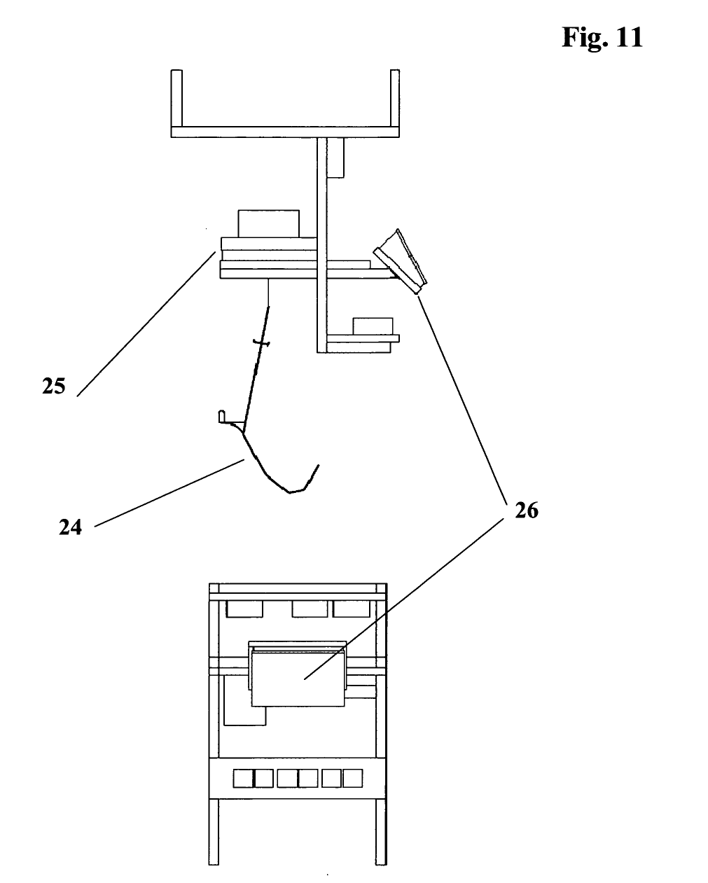

FIG. 11 shows an exemplary application of a support unit (24) as reported herein mounted below a balance (25) in a ceiling mounted rack as side view (top) and as front view (bottom) with control unit (26).

One aspect as reported herein is a cell cultivation apparatus comprising:
a) a cell culture chamber,
b) a gas supply,
c) at least liquid storage units for an acidic solution, a basic solution, an anti-foam solution, and a nutrient solution,
d) at least sensors for determining the concentration of oxygen, the concentration of carbon dioxide, the pH value, the temperature, and the glucose concentration in the cell culture chamber,
e) a control unit for recording the sensor data and controlling the addition of the solutions to the cell culture chamber,
f) at least one pump for the addition of the solution to the cell culture chamber under control of the control unit e),
whereby
up to four storage units are connected to one balance, and
the end of the addition of the solutions to the cell cultivation chamber is triggered by the weight loss of the respective storage unit from the beginning of the addition.

A control of the addition of solutions to a cultivation vessel by weight allows for the control of the addition by a limited number, or even a single, balance, therewith providing for a reduction of the required equipment/technical complexity of the cultivation apparatus. Likewise therewith the accessibility of the cultivation apparatus is improved.

In one embodiment at least one of the storage units connected to a balance is mounted below the balance in a support unit as reported herein. It has been found that an improved support unit has to be used, especially when flexible bag-type cell cultivation liquid storage units made of plastic are used. The support unit as reported herein prevents, e.g. the rupture of the storage unit, especially when large bag-type storage units are used.

As balance any balance can be used as long as that balance has a maximum capacity more than the combined weight of the employed storage units and liquids and as long as the balance has a means for placing a storage unit below it. In another embodiment the balance including the storage units and the control unit is located above the cell culture vessel. The term “A is located above B” denotes that “A” is sitting above “B”, i.e. the distance of the lowest point of A to the floor of the room is more than that of the highest point of B. In one embodiment the lowest point of the balance and the storage units is more than 1.5 m and less than 4.0 m above the floor of the room, in which the cell cultivation apparatus is operated.

Another aspect as reported herein is a support unit for mounting a cell cultivation liquid storage unit, whereby the support unit comprises at one end a first means (1) and at the other end a second means (5), which are connected by a rod having no offset in the first straight section (2) connected with the first means (1) and having one offset section (4) in the second straight section (3) connected with the second means (5). The offset section (4) allows that the center of mass of the entire support unit with the mounted liquid storage unit is positioned in a way that the first straight section (2) connected with the first means (1) is perpendicular to the floor of the room in which the support unit is used when the first means (1) of the support unit with the mounted liquid storage unit is hung up. In one embodiment the offset in the offset section (4) between the axis (101) of the first straight section (2) and the axis (201) of the second straight section (3) is of from 5 to 50 A.U with respect to the first straight section (2) connected with the first means (1).

In another embodiment the offset in the offset section (4) is 10 A.U. The offset section (4) comprises in one embodiment a first bend with an angle of from 90 degrees to 160 degrees, preferably 135 degrees, a straight line connector, preferably of from 8 A.U. to 12 A.U., and a second bend in the opposite direction as the first bend with the same angle as the first bend. The first means (1) of the support unit is in one embodiment an oval eye for taking up a hook and has a length to diameter ratio of about 2:1, preferably with a length of from 10 to 16 A.U. and a diameter of from 5 to 8 A.U. The sections (2) and (3) of the rod have in one embodiment a length of from 72 A.U. to 138 A.U. The second means (5) has an angle of 90 degrees to the direction of the rod in another embodiment. In one embodiment the second means (5) has a distance of from 50 to 60 A.U. to the second bend in the offset section (4).

In one embodiment the support unit for mounting a cell cultivation liquid storage unit is characterized in that the support unit comprises at one end an eye as first means (1) and at the other end a hook as second means (5), which are connected by a rod having no offset in the section (2) connected with the eye (1) and having an offset in the section (3) connected with the hook (5), whereby a) the offset in offset section (4) is 10 A.U. with respect to the rod in the section (2) connected with the eye (1), b) the eye is an oval eye for taking up a hook and has an outer length of 16 A.U. and an outer diameter of 8 A.U., c) the sections (2) and (3) of the rod have a length of 72 A.U. or 138 A.U., d) the offset section (4) comprises i) a first bend with an angle of 135 degrees, a straight line connector of from 8 A.U. to 12 A.U., and a second bend in the opposite directed as the first bend with the same angle as the first bend, and e) the hook (5) has an angle of 90 degrees to the direction of the rod and a distance of from 54 or 58 A.U. to the second bend in the offset section (4).

A further aspect as reported herein is a support unit for mounting a cell cultivation liquid storage unit comprising a central component (6) to which as first to fourth means hooks (7), (701), (8), and (9), respectively, are attached, whereby a) two hooks (7) and (701) are attached in the upper third of the central component (6) opposite to each other at each side of the central component (6), b) a hook (8) is attached in the lower third of the central component (6), and c) a hook (9) is attached to the lower flange (13) of the central component (6).

In one embodiment the central component (6) is in the form of an isosceles trapezoid. The central component (6) has in one embodiment a) a lower flange (13) length of from 290 to 320 A.U., b) an upper flange (14) length of from 90 to 110 A.U., and c) an outside flange (15) length of from 530 to 570 A.U. The hook (7) is in one embodiment a) attached in the upper third of the central component (6), b) of L-shape, and c) attached at an angle of 90 degrees to the central component (6). The hook (8) is in one embodiment attached in the lower third of the central component (6) and has a basis plate (10) in form of a circular segment with a height more than the radius of the corresponding circle, two upward teeth (11) attached at an angle of 90 degree to the basis plate (10), a support plate (12) connected to the bottom of basis plate (10) and to the lower flange (13) of the central component (6), and is attached to the central component (6) with an angle of 75 to 85 degrees. The hook (9) attached to the lower flange (13) of the central component (6) is directed to the opposite side as hook (8), is attached to the lower flange (13) with an angle of 130 to 150 degrees with respect to the central component (6), and is bend three times upward in the direction to the central component (6) with angles of 150 to 165 degrees, 120 to 140 degrees, and 120 to 140 degrees, respectively.

In another embodiment each of the hooks (7) and (701) attached in the upper third of the central component (6) is attached at a distance of 150 to 170 A.U. from the upper flange (14) of the central component (6) and has a basis plate height of 25 to 35 A.U. and an upward height of 10 to 14 A.U. The hook (8) attached in the lower third of the central component (6) has in another embodiment a basis plate (10) height of 120 to 140 A.U., a radius of 90 to 100 A.U., a tooth (11) height of 50 to 60 A.U., a tooth (11) width of 40 to 50 A.U., and the support plate (12) has a length of 70 to 100 A.U. and an angle to the basis plate (10) of 140 to 160 degrees. The hook (9) has in another embodiment a first distance (16) of 180 to 200 A.U. from the lower flange (13) to the first upward bend (20), a second distance (17) of 130 to 150 A.U. from the first upward bend (20) to the second upward bend (21), a third distance (18) of 60 to 75 A.U. from the second upward bend (21) to the third upward bend (22), and a fourth distance (19) of 110 to 130 A.U. from the third upward bend (22) to the end of the hook (23).

In one embodiment the support unit for mounting a cell cultivation liquid storage unit is characterized in that the support unit comprises a central component (6) in the form of a isosceles trapezoid, with a lower flange (13) length of 305 A.U., an upper flange (14) length of 100 A.U., an outside flange (15) length of from 545 to 565 A.U., to which four means (7), (701), (8), and (9) are attached, whereby a) hook (7) and (701) are attached at 160 A.U. from the upper flange (14) of the central component opposite at each side of the central component (6), are of L-shape and attached at an angle of 90 degrees to the central component (6), and have a basis plate height of 30 A.U. and an upward height of 12 A.U., b) a hook (8) in form of a circular segment with two upward teeth (11) attached at an angle of 90 degree to a basis plate (10) and with a support plate (12) connected to the bottom of the basis plate (10) and to the lower flange (13) of the central component (6) that is attached in the lower third of the central component (6) with an angle of about 79 degrees to the central component (6), with the basis plate (10) having a height of 127 A.U., a radius of 94 A.U., a tooth (11) having a height of 55 A.U., a width of 43 A.U., and with a support plate (12) having a length of 89 A.U., a width of 40 A.U. and an angle to the basis plate of 147 degrees, c) a hook (9) is attached to the lower flange (13) of the central component (6) directed to the opposite side as hook (8) with an angle of 140 degrees to the central component (6), and is bend three times upward with angles of 158 degrees, 130 degrees, and 130 degrees, respectively, with a distance (16) of 190 A.U. from the lower flange (13) to the first upward bend (20), a distance (17) of 137 A.U. from the first upward bend (20) to the second upward bend (21), a distance (18) of 67 A.U. from the second upward bend (21) to the third upward bend (22), and a distance (19) of 119 A.U. from the third upward bend (22) to the end of the hook (23).

The term “addition” as used within the current application denotes the adding of a solution of a certain period of time rather than the complete amount all at once. It is also included in this definition a single start and/or a single end or multiple start and/or multiple ends of the addition of the solution.

An isosceles trapezoid is a quadrilateral with a line of symmetry bisecting one pair of opposite sides, making it a trapezoid. Two opposite sides (bases) are parallel, the two other sides (legs) are of equal length. The diagonals are of equal length. An isosceles trapezoid's base angles are congruent. Any quadrilateral with one axis of symmetry must be either an isosceles trapezoid or a kite.

The term “A.U.” denotes an arbitrary unit. With this arbitrary unit for the denotation of sizes it is possible to report support units useful for different sizes of storage units in a single description. The size ratios and the angles are independent of the overall size. In one embodiment 1 A.U. corresponds to 1 mm (millimeter). In one embodiment 1 A.U. corresponds to 1 cm (centimeter). In one embodiment 1 A.U. corresponds to 1 in (inch=2.54 cm).

The following figures are provided to aid the understanding of the present invention, the true scope of which is set forth in the appended claims. It is understood that modifications can be made in the procedures set forth without departing from the spirit of the invention.

1. A support unit for mounting cell cultivation liquid storage units below a carrier unit, characterized in that the support unit comprises:
a)—a planar central component (6) with a plate-like shape with an upper flange (14), a lower flange (13), and a height (302);
b)—a first means (7) and a second means (701) each for mounting a bag-type cell cultivation liquid storage unit;
c)—a third means (8) for mounting a bottle-type cell cultivation liquid storage unit; and
d)—a block-out (301) close to the upper flange (14) of the central component (6) for mounting the support unit to the carrier unit;
whereby
a)—the central component (6) has the shape of an isosceles trapezoid with a width at the upper flange (14) that is less than half the width at the lower flange (13);
b)—the first means (7) and the third means (8) are on the same side of the central component (6), and the first means (7) and the second means (701) are on opposite sides of the central component (6); and,
c)—the first means (7) and the second means (701) are each attached within a distance of at most one third of the height (302) of the central component (6) from the upper flange (14) and the third means (8) is attached within a distance of at most one third of the height (302) of the central component (6) from the lower flange (13).

2. The unit according to claim 1, further characterized in that the unit comprises one or more features selected from the group consisting of
the central component (6) comprising an isosceles trapezoid-like block-out (303),
the first means (7) and the second means (701) having the same shape and size and are attached opposite of each other at each side of the central component (6),
the first means (7) and the second means (701) both having an L-shape with the face (307) that is attached to the central component (6) being perpendicular to the face of the central component (6) and the other face (306) being parallel to the face of the central component (6),
the third means (8) comprising
i) a basis plate (10) with the shape of a segment of a circle with a width (501) of more than the radius of the corresponding circle (502),
ii) two upward teeth (11) at an angle (305) of 90 degree with respect to the basis plate (10), and
iii) a support plate (12) attached i) to the bottom of basis plate (10) and ii) to the lower flange (13) of the central component (6),
whereby the basis plate (10) is attached to the central component (6) with an angle (304) of 75 to 85 degrees, and
the central component (6) further comprising a fourth means (9) that
i) is attached to the lower flange (13) of the central component (6),
ii) is directed to the opposite side of the first means (7) as well as the third means (8), and
iii) is attached to the lower flange (13) with an angle (601) of 130 to 150 degrees, and is bent three times upward with angles (602), (603), and (604) of 150 to 165 degrees, 120 to 140 degrees, and 120 to 140 degrees, respectively.

3. The unit according to claim 1 or claim 2, characterized in that
a)—the central component (6) has
i) a lower flange (13) length of from 290 to 320 A.U.,
ii) an upper flange (14) length of from 90 to 110 A.U.,
iii) an outside flange (15) length of from 530 to 570 A.U.,
b)—the first means (7) and the second means (701) are attached at a distance of 150 to 170 A.U. from the upper flange (14) of the central component (6) and has a basis plate length (307) of 25 to 35 A.U. and an upward height (306) of 10 to 14 A.U.,
c)—the second means (8) attached in the lower third of the central component (6) has a basis plate (10) height of 120 to 140 A.U., a radius of 90 to 100 A.U., a tooth (11) height of 50 to 60 A.U., a tooth width of 40 to 50 A.U., and the support plate (12) has a length of 70 to 100 A.U. and an angle to the basis plate (10) of 140 to 160 degrees, and
d)—the fourth means (9) has a distance (16) of 180 to 200 A.U. from the lower flange (13) to the first upward bend (20), a distance (17) of 130 to 150 A.U. from the first upward bend (20) to the second upward bend (21), a distance (18) of 60 to 75 A.U. from the second upward bend (21) to the third upward bend (22), and a distance (19) 110 top 130 A.U. from the third upward bend (22) to the end of the hook (23).

4. The unit according to claim 1 or claim 2, characterized in that the carrier unit is selected from the group consisting of an electronic balance with a hook at its bottom, a mechanical balance, a moveable scaffold with a fixed height of more than the overall length of the support unit, and a hook.

5. A device comprising the support unit according to claim 1 or claim 2 and a carrier unit selected from the group consisting of an electronic balance with a hook at its bottom, a mechanical balance, and a moveable scaffold with a fixed height of more than the overall length of the support unit.

6. The use of the support unit according to claim 1 or claim 2 for taking up at least three cell cultivation liquid storage units and mounting the support unit to a carrier unit, whereby the support unit is freely suspended below the carrier unit.

7. The unit according to claim 1 or claim 2, characterized in that 1 A.U. is 1 mm, 1 cm, or 1 in.

8. A cell cultivation apparatus comprising:
a) a cell culture chamber,
b) a gas supply,
c) at least cell cultivation liquid storage units for an acidic solution, a basic solution, an anti-foam solution, and a nutrient solution,
d) at least sensors for determining the concentration of oxygen, the concentration of carbon dioxide, the pH value, and the temperature in the cell culture chamber,
e) a control unit for recording the sensor data and controlling the addition of the solutions to the cell culture chamber, and
f) at least one pump for the addition of the solutions of c) to the cell culture chamber under control of the control unit e),
whereby
a)—up to five storage units are connected to one balance,
b)—at least one of the storage units connected to the balance is placed below the balance in a support unit according to claim 1 or claim 2, and
c)—the end of the addition of each of the solutions to the cell cultivation chamber is triggered by the weight loss of the respective liquid storage unit from the beginning of the addition.

9. The apparatus according to claim 8, characterized in that the balance is placed at 1.5 m to 4.0 m above the floor of the room in which the cell cultivation apparatus is operated.

10. The apparatus according to claim 8, characterized in that the liquid storage units are connected via flexible, plastic pipes with the pump and the pump is connected with the cell culture chamber via a flexible, plastic pipe.

11. A method for the cultivation of cells using the cell cultivation apparatus according to claim 8 or claim 9.

12. A method for producing of a polypeptide comprising the following steps:
a) providing a cell containing a nucleic acid encoding the polypeptide,
b) cultivating the cell in a cell cultivation apparatus according to claim 8 or claim 9,
c) recovering the polypeptide from the cultivation medium or the cells, and
d) optionally purifying the polypeptide,
thereby producing a polypeptide.
